(19) United States
(12) Patent Application Publication
(10) Pub. No.: US 2005/0215555 A1
(43) Pub. Date: Sep. 29, 2005

(54) Inhibitors of 5-HT2A receptor

(76) Inventors: Roger Crossley, Sittingbourne (GB); Terry Ward, Coleville (GB); Malin Berthold, Djursholm (SE)

Correspondence Address: FISH & RICHARDSON PC, P.O. BOX 1022, MINNEAPOLIS, MN, 55440-1022, US

(21) Appl. No.: 10/947,998

(22) Filed: Sep. 23, 2004

Related U.S. Application Data
(63) Continuation of application No. 10/933,922, filed on Sep. 2, 2004.
(60) Provisional application No. 60/505,337, filed on Sep. 23, 2003.

(30) Foreign Application Priority Data
Sep. 3, 2003 (SE) 0302368.6

(51) Int. Cl. C07D 471/02; A61K 31/498; A61K 31/4745

(52) U.S. Cl. 514/249; 514/303; 544/350; 546/118

(57) ABSTRACT

Compounds of Formula (I): 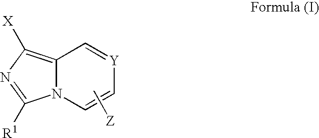
wherein X, Y, Z, and R¹ are as described herein, processes for preparing the compounds, pharmaceutical compositions comprising the compounds, and use of the compounds and compositions in the prophylaxis or treatment of a 5-HT_{2A} receptor-related disorder.

### CROSS REFERENCE TO RELATED APPLICATIONS

This application claims priority to U.S. application Ser. No. 10/933,922, filed Sep. 2, 2004; U.S. provisional application Ser. No. 60/505,337, filed Sep. 23, 2003; and to Swedish Patent Application No. 0302368.6, filed Sep. 3, 2003. The prior applications are incorporated herein by reference in their entirety.

### TECHNICAL FIELD

The present invention relates to novel compounds, to pharmaceutical compositions comprising the compounds, to processes for their preparation, as well as to the use of the compounds for the preparation of a medicament against 5-HT_{2A} receptor-related disorders.

### BACKGROUND

Many disorders and conditions of the central nervous system are influenced by the adrenergic, the dopaminergic, and the serotonergic neurotransmitter systems. For example, serotonin (5-HT; 5-hydroxytryptamine) has been implicated in a number of disorders and conditions which originate in the central nervous system.

The 5-HT_{2A} receptor has been implicated as a therapeutic target for the treatment or prevention of abnormalities of the serotonergic system, including psychotic disorders such as schizophrenia (A. Carlsson, N. Waters and M. L. Carlsson, Biol. Psychiatry, 46, 1388-1395 (1999); G. J. Marek and G. K. Aghajanian, Biol. Psychiatry, 44, 1118-1127 (1998); E. Sibelle, Z. Sarnyai, D. Benjamin, J. Gal, H. Baker and M. Toth, Mol. Pharmacol., 52, 1056-1063 (1997)). Abnormality of this system has also been implicated in a number of human diseases such as mental depression (Arias B, Gutierrez B, Pintor L, Gasto C, Fananas L, Mol. Psychiatry (2001) 6, 239-242), migraine, epilepsy and obsessive-compulsive disorder (Luisa de Angelis, Curr. Opin. Invest. Drugs (2002) 3 (1) 106-112). 5-HT_{2A} antagonists may also be useful in the treatment of sleep disorders such as insomnia and obstructive sleep apnea, anorexia nervosa (Ziegler A, Gorg T, Lancet (1999) 353, 929), cardiovascular conditions such as hypertension, vasospasm, angina, Raynaud's phenomenon and thrombotic illness including stroke, glaucoma (T. Mano et al. and H. Takaneka et al., Invest. Ophthamol. Vis. Sci., 1995, vol. 36, pages 719 and 734, respectively) and in the inhibition of platelet aggregation. Evidence also implies that selective 5-HT_{2A} receptor antagonists may also be useful in the treatment of alcohol and cocaine dependence (Maurel S, De Vry J, De Beun R, Schreiber, Pharmacol. Biochem Behav (1999) 89-96; McMahon L R, Cunningham K A, J. Pharmacol Exp Ther (2001) 297, 357-363).

No publications disclose the use of the compounds according to the present invention against 5-HT_{2A} receptor-related disorders.

### SUMMARY

One object of the present invention is a compound of the Formula (I) 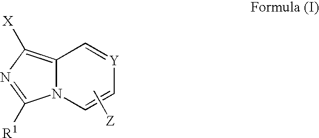
wherein

X is selected from aryl and heteroaryl, optionally independently substituted with one or more of C₁₋₆-alkyl, C₁₋₆-alkoxy, methylenedioxy, aryl, halogen, and halo-C₁₋₆-alkyl;

Y is selected from C-Z and N;

Z is selected from hydrogen, C₁₋₆-alkyl, C₁₋₆-alkoxy, and halogen;

R¹ is either a group 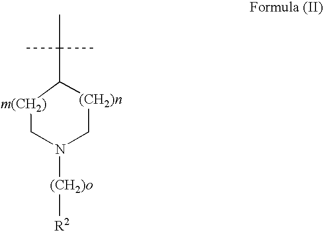
wherein
R² is either
hydrogen; or
C₂₋₆-alkenyl, provided that o is 1; or
aryl optionally independently substituted with one or more of C₁₋₆-alkoxy, halogen, cyano, and methylenedioxy, provided that o is 1-3; or
aryl-C₁₋₆-alkyl provided that o is 0; or
aryloxy optionally independently substituted with one or more of C₁₋₆-alkoxy and halogen, provided that o is 2-3; or
heteroaryl optionally independently substituted with one or more of C₁₋₆-alkyl and C₁₋₆-alkoxy; or
heterocyclyl optionally independently substituted with one or more of C₁₋₆-alkyl and C₁₋₆-alkoxy;

m is 0 or 1;

n is 1 or2;

o is 0, 1, 2, or 3; or

R¹ is a group 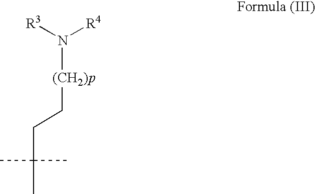
wherein

R³ is hydrogen or C₁₋₆-alkyl;

R⁴ is C₁₋₆-alkyl, aryl optionally independently substituted with one or more of C₁₋₆-alkyl and C₁₋₆-alkoxy; or heteroaryl-C₁₋₆-alkyl;

p is 0 or 1; and

pharmaceutically acceptable salts, hydrates, solvates, geometrical isomers, tautomers, optical isomers, and prodrug forms thereof.

It is preferred that X is selected from
phenyl, optionally independently substituted with one or more of methyl, methoxy, methylenedioxy, phenyl, chloro, fluoro, and trifluoromethyl; and
thienyl.

It is even more preferred that X is selected from phenyl, 3-methylphenyl, 2-methoxyphenyl, 3-methoxyphenyl, 4-methoxyphenyl, 3,4-methylenedioxyphenyl, 1,1′-biphenyl-4-yl, 4-chlorophenyl, 4-fluorophenyl, 2-thienyl, and 4-trifluoromethylphenyl.

It is preferred that Z is selected from hydrogen, methyl, chloro, and methoxy.

It is preferred that R² is selected from
hydrogen;
vinyl;
indanyl;
phenyl, optionally independently substituted with one or more of methyl, methoxy, bromo, fluoro, cyano, and methylenedioxy;
phenylethyl;
phenoxy, optionally independently substituted with one or more of methoxy, fluoro, chloro, and bromo;
indolyl, optionally independently substituted with one or more of methoxy;
thienyl; and
hexahydro-1H-isoindole-1,3(2H)-dione.

It is even more preferred that R² is selected from hydrogen, vinyl, phenyl, 2-indanyl, 3-methylphenyl, 3,4,5-trimethoxyphenyl, 4-bromophenyl, 4-fluorophenyl, 1-phenylethyl, phenoxy, 2,6-dimethoxyphenoxy, 4-fluorophenoxy, 3-indolyl, 5-methoxy-3-indolyl, 2-thienyl, and hexahydro-1H-isoindole-1,3(2H)-dione.

It is preferred that m+n is 1 or 2.

It is preferred that R³ is selected from hydrogen and methyl.

It is preferred that R⁴ is selected from methyl, 2-indanyl, and 2-methyl-3-(3,4-methylenedioxyphenyl)-n-propyl.

Preferred compounds are given in Examples 1-40.

Another object of the present invention is a process for the preparation of a compound as mentioned above, which process comprises the following steps:

a) reaction of a compound of Formula (IV)

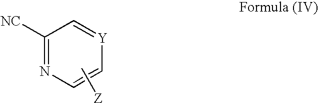

wherein

Y is selected from C—Z and N;

Z is selected from hydrogen, C₁₋₆-alkyl, C₁₋₆-alkoxy, and halogen;

with a Grignard reagent of Formula X—MgBr and then reduction with a reducing agent such as sodium borohydride

wherein

X is selected from aryl and heteroaryl, optionally independently substituted with one or more of C₁₋₆-alkyl, C₁₋₆-alkoxy, methylenedioxy, aryl, halogen, and halo-C₁₋₆-alkyl;

to give a compound of Formula (V)

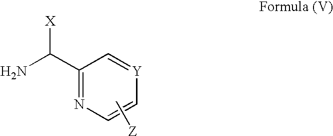

wherein X, Y, and Z are as defined above,

b) amidation_by reaction of the compound of Formula (V) with either a carboxylic acid of Formula (VI) or of Formula (VII) in the presence of a coupling agent such as carbonyldiimidazole

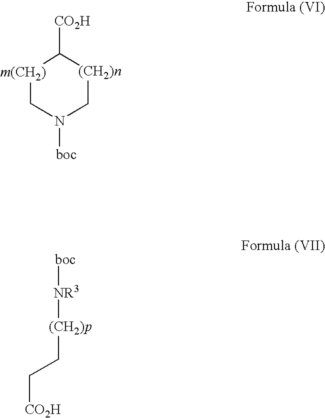

wherein

m is 0 or 1;

n is 1 or 2;

p is 0 or 1;

R³ is hydrogen or C₁₋₆-alkyl;

to give a compound of Formula (VIII) and (IX), respectively,

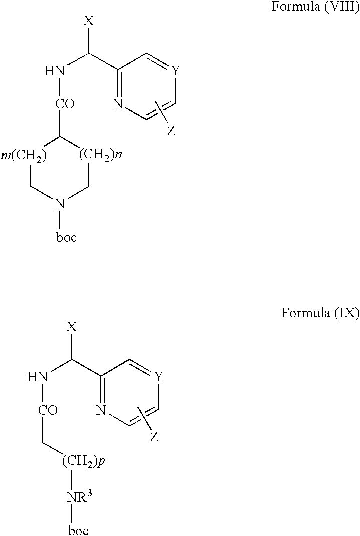

wherein X, Y, Z, m, n, p, and R³ are as defined above,

c) cyclization of the compound of Formula (VEII) with phosphorous oxychloride or the compound of Formula (IX) with trifluoroacetic anhydride, respectively, to give a compound of Formula (X) or (XI), respectively,

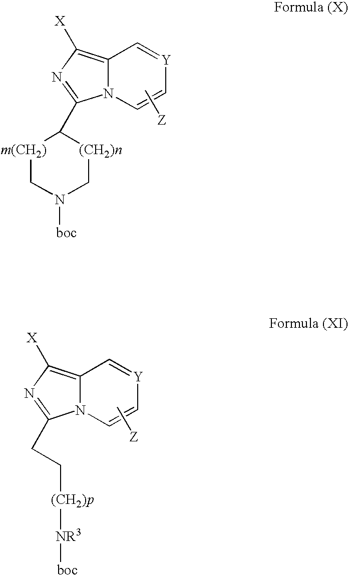

wherein X, Y, Z, m, n, p, and R³ are as defined above,

d) deprotection of the compound of Formula (X) or (XI), respectively, under acidic conditions, to give compounds of Formula (XII) or (XIII), respectively,

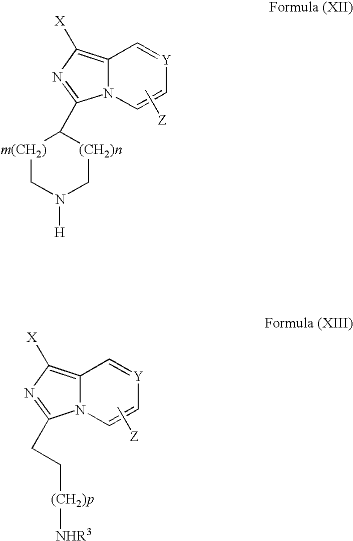

wherein X, Y, Z, m, n, p, and R³ are as defined above; and, optionally, either of steps e) and f)

e) alkylation of the compound of Formula (XII) or (XIII), respectively, via displacement of a leaving group according to e1) and e2):

e1) reaction of the compound of Formula (XII) with an alkylating agent of the Formula R²—(CH₂)^{o}-LG in the presence of a tertiary amine such as N-ethyldiisopropylamine, wherein R² is selected from C₂₋₆-alkenyl, provided that o is 1; aryl optionally independently substituted with one or more of C₁₋₆-alkyl, C₁₋₆-alkoxy, halogen, cyano, and methylenedioxy, provided that o is 1-3; ary -C₁₋₆-alkyl, provided that o is 0; aryloxy optionally independently substituted with one or more of C₁₋₆-alkoxy and halogen, provided that o is 2-3; heteroaryl optionally independently substituted with one or more of C₁₋₆-alkyl and C₁₋₆-alkoxy; or heterocyclyl optionally independently substituted with one or more of C₁₋₆-alkyl and C₁₋₆-alkoxy, o is 0, 1, 2, or 3, and LG is a leaving group, to give a compound of Formula (XIV); or

e2) reaction of the compound of Formula (XIII) with an alkylating agent of the Formula R⁴-LG in the presence of a base (e.g., N-ethyldiisopropylamine), wherein R⁴ is aryl optionally independently substituted with one or more of C₁₋₆-alkyl and C₁₋₆-alkoxy, or heteroaryl-C₁₋₆-alkyl; and LG is as defined above, to give a compound of Formula (XV)

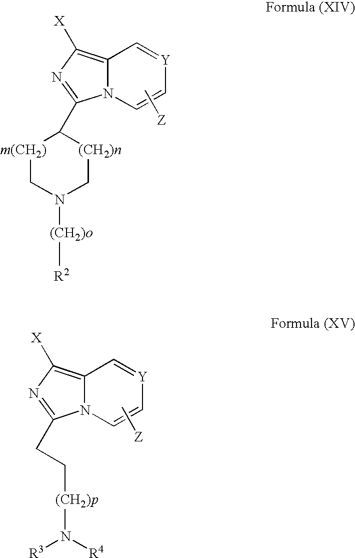

wherein X, Y, Z, m, n, o, p, R², R³, and R⁴ are as defined above;

f) alkylation of the compound of Formula (XII) or (XIII), respectively, via reductive amination according to f1) and f2):

f1) reaction of the compound of Formula (XII) with an aldehyde of the formula R²—(CH₂)_{q}—CHO, wherein R² is as defined above and q is 1-2, acetophenone or 2-indanone then a reducing agent such as sodium triacetoxyborohydride, to give a compound of Formula (XIV); or

f2) reaction of the compound of Formula (XII) with an aldehyde of the formula R⁵—CHO, wherein R⁵ is heteroaryl-C₁₋₆-alkyl, preferably 1-methyl-2-(3,4-methylenedioxyphenyl)ethyl, or 2-indanone and then a reducing agent such as sodium triacetoxyborohydride, to give a compound of Formula (XV).

Another object of the present invention is a compound as mentioned above for use in therapy, especially for use in the prophylaxis or treatment of a 5-HT_{2A} receptor-related disorder.

Another object of the present invention is a pharmaceutical formulation comprising a compound as mentioned above as active ingredient, in combination with a pharmaceutically acceptable diluent or carrier, especially for use in the prophylaxis or treatment of a 5-HT_{2A} receptor-related disorder.

Another aspect of the present invention is a method for treating a human or animal subject suffering from a 5-HT_{2A} receptor-related disorder. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the 5-HT_{2A} receptor-related disorder. Identifying a subject in need of such treatment can be in the judgment of a subject or a health care professional and can be subjective (e.g., opinion) or objective (e.g., measurable by a test or diagnostic method).

Another object of the present invention is a method for the prophylaxis of a 5-HT_{2A} receptor-related disorder, which comprises administering to a subject in need of such treatment an effective amount of a compound as mentioned above.

Another object of the present invention is a method for modulating 5-HT_{2A} receptor activity, which comprises administering to a subject in need of such treatment an effective amount of a compound as mentioned above.

Another object of the present invention is the use of a compound as mentioned above for the manufacture of a medicament for use in the prophylaxis or treatment of a 5-HT_{2A} receptor-related disorder.

The compounds as mentioned above may be agonists, partial agonists or antagonists for the 5-HT_{2A} receptor. Preferably, the compounds of the present invention act as 5-HT_{2A} receptor antagonists. More preferably, the compounds of the present invention act as selective 5-HT_{2A} receptor antagonists.

Examples of 5-HT_{2A} receptor-related disorders are schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence.

The compounds and compositions are useful for treating diseases, including schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence. In one aspect, the invention relates to a method for treating or preventing an aforementioned disease comprising administrating to a subject in need of such treatment an effective amount of a compound or composition delineated herein.

The following definitions shall apply throughout the specification and the appended claims.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted with one or more halogen atoms. Likewise, “aryl-C₁₋₆-alkyl” means a C₁₋₆-alkyl group substituted with one or more aryl groups.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “C₂₋₆-alkenyl” denotes a straight or branched alkenyl group having from 2 to 6 carbon atoms. Examples of said alkenyl include vinyl, allyl, 1-butenyl, 1 -pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₃₋₅-alkenyl, C₃₋₄-alkenyl, C₄₋₆-alkenyl, C₄₋₅-alkenyl, etc.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring. Examples of aryls are phenyl, pentalenyl, indenyl, indanyl, isoindolinyl, chromanyl, naphthyl, fluorenyl, anthryl, phenanthryl and pyrenyl. The aryl rings may optionally be substituted with C₁₋₆ -alkyl. Examples of substituted aryl groups are 2-methylphenyl and 3-methylphenyl. Likewise, “aryloxy” refers to an aryl group bonded to an oxygen atom.

The term “heteroaryl” refers to a hydrocarbon ring system having at least one aromatic ring having one or more ring atoms that are a heteroatom such as O, N, or S, and the remaining ring atoms are carbon. Examples of heteroaryl groups include furyl, pyrrolyl, thienyl, oxazolyl, isoxazolyl, imidazolyl, thiazolyl, isothiazolyl, pyridinyl, pyrimidinyl, quinazolinyl, indolyl, pyrazolyl, pyridazinyl, quinolinyl, benzofuranyl, dihydrobenzofuranyl, benzodioxolyl, benzodioxinyl, benzothiazolyl, benzothiadiazolyl, and benzotriazolyl groups.

The term “heterocyclyl” refers to a hydrocarbon ring system containing 4 to 8 ring members that have at least one heteroatom (e.g., S, N, or O ) as part of the ring. It includes saturated, unsaturated, and nonaromatic heterocycles. Suitable heterocyclic groups include the above-mentioned heteroaryl groups, pyrrolidinyl, piperidyl, azepinyl, morpholinyl, thiomorpholinyl, pyranyl, dioxanyl, and hexahydro-1H-isoindole-1,3(2H)-dione groups.

The term “leaving group” refers to a group to be displaced from a molecule during a nucleophilic displacement reaction. Examples of leaving groups are bromide, chloride methanesulfonate, hydroxy, methoxy, thiomethoxy, tosyl, or suitable protonated forms thereof (e.g., H₂O, MeOH), especially bromide and methanesulfonate.

The term “reducing agent” refers to a substance capable of reducing another substance and it itself is oxidized. Examples of reducing agents include, but are not limited to, hydrogen, sodium, potassium, sodium borohydride, sodium cyanoborohydride, sodium triacetoxyborohydride, lithium aluminiumhydride, and diisobutylaluminium hydride. “Coupling agent” refers to a substance capable of catalyzing a coupling reaction, such as amidation, or esterification. Examples of coupling agents include, but are not limited to, carbonyldiimidazole, dicyclohexylcarbodimide, pyridine, 4-dimethylaminopyridine, and triphenylphosphine.

“Acidic condition” refers to a reaction condition in which the reaction is carried out in the presence of a certain amount of acid. Examples of acids include, but are not limited to, inorganic acids, such as hydrogen chloride, hydrogen bromide, hydrogen iodide, sulfuric acid, nitric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. “Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

The following abbreviations have been used:

ACN means acetonitrile,

AP means atmospheric pressure chemical ionisation,

BOC means tert-butoxycarbonyl,

(Boc)₂O means di-tert-butyl dicarbonate,

CDI means carbonyldiimidazole,

DCM means dichloromethane,

DEA means diethylamine,

DEPT means distortion enhancement polarization transfer,

DIBAL-H means diisobutylaluminium hydride,

DMF means dimethylformamide,

DMSO means dimethyl sulfoxide,

DPAT means dipropylaminotetraline,

HPLC means high performance liquid chromatography,

Hunig's base means N-ethyldiisopropylamine,

MIBK means methyl isobutylketone,

POCl₃ means phosphorous oxychloride,

QC means quality control

Rt means retention time,

TEA means triethylamine,

TFA means trifluoroacetic acid,

TFAA means trifluoroacetic anhydride,

THF means tetrahydrofuran,

TLC means thin layer chromatography.

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z- double bond isomer forms. All isomeric forms are contemplated.

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd}Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body_weight, administered singly or multiply in doses, e.g. from about 0.01 mg to about 25 mg each. Normally, such a dosage is given orally but parenteral administration may also be chosen.

The details of one or more embodiments of the invention are set forth in the accompanying drawings and the description below. Other features, objects, and advantages of the invention will be apparent from the description and drawings, and from the claims.

### DETAILED DESCRIPTION

In the examples described below, all reagents were commercial grade and were used as received without further purification, unless otherwise specified. The chemicals were bought from Sigma-Aldrich (The old brickyard, New road, Gillingham, Dorset, SP8 4XT, UK), Lancaster (Eastgate, White Lund, Morecambe, Lancashire, LA3 3DY, UK), and Acros (Bishop Meadow road, Loughborough, leicestershire, LE11 5RG, UK). Commercially available anhydrous solvents were used for reactions conducted under inert atmosphere. Reagent grade solvents were used in all other cases, unless otherwise specified. Column chromatography was performed on Matrex® silica gel 60 (35-70 micron). TLC was carried out using pre-coated silica gel F-254 plates (thickness 0.25 mm). ¹H NMR spectra were recorded on a Bruker Avance250 at 250 MHz. Chemical shifts for ¹H NMR spectra are given in part per million and either tetramethylsilane (0.00 ppm) or residual solvent peaks were used as internal reference. Splitting patterns are designated as follows: s, singlet; d, doublet; t, triplet; q, quartet; p, pentet; m, multiplet; br, broad. Coupling constants are given in Hertz (Hz). Only selected data are reported. The ¹³C NMR spectra were recorded at 62.5 MHz. DEPT experiments were used to help assign ¹³C NMR resonances where necessary. Chemical shifts for ¹³C NMR spectra are expressed in parts per million and residual solvent peaks were used as internal reference. HPLC analyses were performed using a Waters Xterra MS C18 column (100×4.6 mm, 5μ) eluting with a gradient of 5% ACN in 95% water to 95% ACN in 5% water (0.2% TFA buffer) over 3.5 mins, then 95% ACN in 5% water (0.2% TFA buffer) for a further 2.5 mins at a flow rate of 3 ml/min on a Waters 600E or Gilson system with monitoring at 254 nm. Reverse phase preparative HPLC was carried out using a Xterra MS C18 column (100×19 mm, 5 μm) eluting with a gradient of 5% ACN in 95% water to 95% ACN in 5% water (0.05% DEA) over 12.0 mins, then 95% ACN in 5% water (0.05% DEA) for a further 5.0 mins at a flow rate of 25 ml/min with monitoring at 254 nm. The fractions that contained the desired product were concentrated under reduced pressure and the resultant residue was lyophilised from a mixture of dioxane and water. Electrospray MS spectra were obtained on a Micromass platform LCMS spectrometer. Compounds were named using AutoNom 2000.

### EXAMPLE 1

### 2-(3-{4-[1-(4-fluorophenyl)imidazo[1,5-a]pyridin-3-yl]piperidin-1-yl}propyl)hexahydro-1H-isoindole-1,3(2H)-dione

To a solution of the 1-(4-fluorophenyl)-3-piperidin-4-ylimidazo[1,5-a]pyridine (synthesized according to General procedure A; step 1-4) (157 mg, 0.53 mmol) and Hunig's base (276 μl, 0.58 mmol) in dry acetonitrile (12 ml) and dry methanol (1 ml) was added 3-(1,3-dioxooctahydro-2H-isoindol-2-yl)propyl methanesulfonate (167 mg, 0.58 mmol). The reaction mixture was heated to 100° C. for 18 hrs. The reaction mixture was evaporated and the crude was diluted with water and extracted with AcOEt. The organic layers were combined washed with brine, dried over MgSO₄ and concentrated. The residue was purified by flash chromatography on silica gel eluting with a mixture of AcOEt/methanol (10:0) to (9:1) and afforded the desired product (47 mg, 18%) as a brown solid.

¹H-NMR (250 MHz, MeOD): 1.28-1.52 (m, 4H, CH), 1.68-1.98 (m, 5H, CH), 2.01-2.13 (m, 2H, CH), 2.14-2.19 (m, 3H, —CH), 2.41-2.49 (m, 2H, CH), 2.48-2.57 (m, 2H, CH), 2.76-2.92 (m, 2H, CH), 2.94-3.12 (m, 3H, CH), 3.39-3.57(m, 2H, CH), 6.55 (dd, 1H, J=7.5 Hz, J=5 Hz, Harom), 6.70 (dd, 1H, J=5 Hz, Harom), 7.02-7.18 (m, 2H, Harom), 7.63-7.86 (m, 4H, Harom), HPLC 100%, Rt=1.94 min. MS (AP) m/z 489.33 (M+H).

### EXAMPLE 2 (GENERAL PROCEDURE A)

### 1-phenyl-3- [1-(2-phenylethyl)piperidin-4-yl]imidazo [1,5-al pyridine

Step 1

1-Phenyl-1-pyridin-2-ylmethanamine

A solution of 2-cyanopyridine (1 g, 9.6 mmol) in dry toluene (30 mL) under nitrogen was cooled to 0° C. The phenylmagnesium bromide (3.53 ml, 10.6 mmol) was added dropwise over 30 min and the reaction mixture was warmed up to room temperature and stirred for 1 h. The reaction mixture was then cooled down to 0° C. and isobutanol (12 mL) was added dropwise keeping the temperature below 5° C. The reaction mixture was cooled to 0-5° C. and sodium borohydride (510 mg, 13.5 mmol) was added portionwise. The reaction mixture was allowed to warm up to room temperature and stirred overnight. The reaction mixture was quenched with methanol/water and concentrated in vacuo to remove the toluene. The mixture was extracted with DCM and the organic layers dried over magnesium sulphate were concentrated under vacuum to yield the desired amine as a yellow oil (2 g crude). The amine was used without further purification.

¹H-NMR(250 MHz, CDCl₃) δ=2.33 (br, 2H, NH₂), 5.26 (s, 1H, CH), 7.12-7.61 (m, 8H, Harom), 8.58 (d, 1H, J=5 Hz, Harom). ¹³C-NMR(62.5 MHz, DMSO-d₆) δ=61.0, 121.6, 121.9, 127.0, 127.2, 128.6, 136.6, 144.6, 149.1, 163.3. HPLC 92.7%, Rt=1.35 min. MS (AP) m/z 184.05 (M+H).

Step 2

4-[(1-Phenyl-pyridin-2-yl-methyl)-carbamoyl]piperidine-1-carboxylic acid tert-butyl ester

To a stirred solution of Boc-isonipecotic acid (1.7 g, 13.8 mmol) in dry DCM (50 mL) was added a suspension of CDI (2.23 g, 13.8 mmol) in DCM (20 mL). The reaction mixture was stirred for 30 min. A solution of 1-phenyl-1-pyridin-2-ylmethanamine from Step 1 (1.7 g, 9.2 mmol) in dry DCM (50 mL) was then added and the reaction mixture was stirred overnight at room temperature. The reaction mixture was extracted with DCM, washed with water. The organic layers were combined and dried over magnesium sulphate then concentrated in vacuo to yield the desire amide as a yellow powder (3.3 g, 91%).

¹H-NMR(250 MHz, CDCl₃) δ=1.46 (s, 9H, tBu), 1.55-1.75 (m, 2H, 2-CH), 1.79-1.93 (m, 2H, 2-CH), 2.31-2.39 (m, 1H, CH), 2.68-2.87 (m, 2H, 2-CH), 4.07-4.23 (m, 2H, 2-CH), 6.12 (d, 1H, J=7.5 Hz, CHNH), 7.17-7.32 (m, 6H, Harom), 7.62 (dt, 1H, J1=7.5 Hz, J2=2.5 Hz, Harom), 7.75 (brd, 1H, J1=7.5 Hz, Harom), 8.57 (dd, 1H, J1=5 Hz, J2=2.5 Hz Harom), HPLC 99%, Rt=1.97 min. MS (AP) m/z 396.19 (M+H).

Step 3

4-(1-Phenyl-imidazo[1,5-a]pyridin-3-yl)-piperidine-1-carboxylic acid tert-butyl ester

To a cooled (ice/water) solution of the amide (300 mg, 0.76 mmol) and pyridine (380 μl, 4.7 mmol) in DCM (5 ml) was added dropwise POCl₃ (84 μl, 0.9 mmol). The reaction mixture was stirred overnight at room temperature. The mixture was washed with water and extracted with DCM. The organics were dried over magnesium sulfate and concentrated in vacuo to yield the desired cyclised product (257 mg, 89%). The compound was used without further purification.

¹H-NMR(250 MHz, CDCl₃) δ=1.48 (s, 9H, tBu), 1.95-2.11 (m, 4H, 4-CH), 2.89-3.08 (m, 2H, 2-CH), 3.12-3.27 (m, 1H, CH), 4.25-4.38 (m, 2H, 2-CH), 6.53 (dt, 1H, J1=5 Hz, J2=2.5 Hz, Harom), 6.72 (dt, 1H, J1=5 Hz, J2=2.5 Hz, Harom), 7.43 (dt, 2H, J1=7.5 Hz, J2=2.5Hz Harom), 7.74 (dd, 2H, J1=7.5 Hz, J2=2.5 Hz, Harom), 7.85 (dd, 2H, J1=7.5 Hz, J2=2.5 Hz, Harom), 8.57 (brd, 1H, J=5 Hz, Harom), HPLC 100%, Rt=2.15 min. MS (AP) m/z 378.18 (M+H).

Step 4

1-Phenyl-3-piperidine-4-yl-imidazo [1,5-a ]pyridine

To a solution of 4-(1-Phenyl-imidazo[1,5-a]pyridin-3-yl)-piperidine-1-carboxylic acid tert-butyl ester (1.0 g, 2.65 mmol) in dry methanol (1 ml) was added a 4M solution of HCl in dioxane (5.3 mL, 21.0 mmol). The reaction mixture was stirred for 4 hrs at room temperature. The solvent was removed in vacuo and the solid residue triturated with diethyl ether. The solid was removed by filtration and dried to give the amine hydrochloride. The compounds were stored as the HCl salt (831 mg, 100%).

¹ H-NMR(250 MHz, CDCl₃) δ=2.31-2.42 (m, 2H, 2-CH), 2.61-2.80 (m, 2H, 2-CH), 2.81-2.93 (m, 2H, 2-CH), 3.18-3.27 (m, 1H, CH), 3.51-3.70 (m, 2H, 2-CH), 6.51 (dt, 1H, J1=5 Hz, J2=2.5 Hz, Harom), 6.72 (dt, 1H, J1=5 Hz, J2=2.5 Hz, Harom), 7.44 (dt, 2H, J1=7.5 Hz, J2=2.5 Hz Harom), 7.81 (dd, 2H, J1=7.5 Hz, J2=2.5 Hz, Harom), 7.96 (dd, 2H, J1=7.5 Hz, J2=2.5 Hz, Harom), 8.57 (brd, 1H, J=5 Hz, Harom), HPLC 82%, Rt=1.47 min. MS (AP) m/z 278.12 (M+H).

Step 5

To a solution of 1-phenyl-3-piperidin-4-yl-imidazo [1,5-a]pyridine (365 mg, 1.32 mmol) and Hunig's base (574 μl, 3.3 mmol) in dry acetonitrile (10 ml) was added 2-(bromoethyl)benzene (150μl, 1.10 mmol). The reaction mixture was heated to 100° C. for 14hrs. The reaction mixture was evaporated and the crude product was diluted with water and extracted with AcOEt. The organic layers were combined washed with water, dried over MgSO₄ and concentrated. The residue was purified by flash chromatography on silica gel eluting with a mixture of hexane/AcOEt (3:7) to (0:10) followed by AcOEt/methanol (10:0) to (9:1) and afforded the desired compound (178 mg, 36%) as a yellow solid.

¹ H-NMR (250 MHz, CDCl₃): 2.28-2.41 (m, 2H, 2-CH), 2.73-2.89 (m, 2H, 2-CH), 3.17-3.38 (m, 5H, 5-CH), 3.62-3.84 (m, 4H, 4-CH), 6.62 (dd, 1H, J=7.5 Hz, J=2.5 Hz, Harom), 6.80 (dd, 1H, J=7.5 Hz, J=2.5 Hz, Harom), 7.28-7.36 (m, 6H, Harom), 7.48 (dd, 2H, J=7.5 Hz, J=2.5 Hz, Harom), 7.86 (dd, 4H, J1=7.5 Hz, J2=5 Hz, Harom), HPLC 100%, Rt=1.90 min. MS (AP) m/z 382.33 (M+H).

### EXAMPLE 3

### 3-{1-[2-(4- fluorophenyl)ethyl]piperidin 4-yl}-1-(3-methoxyphenyl)imidazo[1,5-a]pyridine

To a solution of 1-(3-methoxy-phenyl)-3-piperidin-4-yl-imidazo [1,5-a]pyridine (synthesized according to General procedure A; step 1-4) (100 mg, 0.32 mmol) and Hunig's base (169 μl, 0.97 mmol) in dry acetonitrile (5 ml) and dry methanol (1 ml) was added methanesulfonic acid 2-(4-fluorophenyl)-ethyl ester (71 mg, 0.325 mmol). The reaction mixture was heated to 100° C. for 2 days. The reaction mixture was evaporated and the crude was diluted with water and extracted with AcOEt. The organic layers were combined washed with water, dried over MgSO₄ and concentrated. The residue was purified by flash chromatography on silica gel eluting with a mixture of hexane/AcOEt (3:7) to (0:10) followed by AcOEt/methanol (10:0) to (9:1) and afforded the desired product (14 mg, 10%) as a brown solid.

¹ H-NMR (250 MHz, MeOD): 0.81-0.93 (m, 4H, 2-CH₂), 1.63-1.76 (m, 4H, CH₂), 1.78-1.91 (m, 2H, CH₂), 2.07-2.19 (m, 1H, —CH), 2.18-2.27 (m, 2H, CH₂), 4.11 (s, 3H, OCH₃), 5.35 (dd, 1 H, J=7.5 Hz, Harom), 5.49 (dd, 2H, J=7.5 Hz, Harom), 5.65 (dd, 2H, 2Harom), ), 5.90-6.00 (m, 5H, 5Harom), 6.43 (d, 1H, J=7.5 Hz, Harom), 6.82-6.79 (d, 1H, J1=5 Hz, Harom), HPLC 98%, Rt=2.04 min. MS (AP) m/z 430.29 (M+H).

### EXAMPLE 4

### 7-methyl-1-phenyl-3-[1-(2-phenylethyl)piperidin-4-yl]imidazo[1,5-a]pyridine

To a solution of 7-methyl-3-(piperidin-4-yl)-1-phenyl-imidazo[1,5-a]pyridine (synthesized according to General procedure A; step 1-4) (1 g, 3.43 mmol) and Hunig's base (3.59 ml, 20.6 mmol) in dry acetonitrile (10 ml) and dry methanol (2 ml) was added 2-(bromoethyl)benzene (468 μl, 3.43 mmol). The reaction mixture was heated to 100° C. over the weekend. The reaction mixture was evaporated and the crude was diluted with water and extracted with AcOEt. The organic layers were combined washed with water, dried over MgSO₄ and concentrated. The residue was purified by flash chromatography on silica gel eluting with a mixture of AcOEt/hexane (8:2) to (10:0) and afforded the desired product (25.2 mg, 2%) as a brown gum.

¹H-NMR (250 MHz, MeOD): 1.72-1.89 (m, 2H, 2-CH), 1.90-2.04 (m, 2H, 2-CH), 2.05-2.22 (m, 2H, 2-CH), 2.34 (s, 3H, CH₃), 2.54-2.73 (m, 2H, 2-CH), 2.74-2.92 (m, 2H, 2-CH), 2.92-3.04 (m, 2H, 2-CH), 3.13-3.25 (m, 1H, 1-CH), 6.37 (d, 1H, J=7.5 Hz, Harom), 7.15-7.32 (m, 6H, 6Harom), 6.89 (dd, 2H, J=7.5 Hz, 2Harom), 7.52 (s, 1H, Harom), 7.74 (dd, 1H, J=7.5 Hz, Harom), 7.86 (dd, 2H, J1=7.5 Hz, J2=2.5Hz, Harom), HPLC 89%, Rt=1.97 min. MS (AP) m/z 396.32 (M+H).

### EXAMPLE 5

### 1-(4-chlorophenyl)-3-[1-(2-phenylethyl)piperidin4-yl]imidazo[1,5-a]pyridine

To a solution of 1-(4-chloro-phenyl)-3-piperidin-4-yl-imidazo[1,5-a]pyridine (synthesized according to General procedure A; step 1-4) (100 mg, 0.32 mmol) and Hunig's base (140μl, 0.8 mmol) in dry acetonitrile (2 ml) and dry methanol (2 ml) was added 2-(bromoethyl)benzene (36.5μl, 0.267 mmol). The reaction mixture was heated to 100° C. for 18hrs. The reaction mixture was evaporated and the crude was diluted with water and extracted with AcOEt. The organic layers were combined washed with water, dried over MgSO₄ and concentrated. The residue was purified by flash chromatography on silica gel eluting with a mixture of hexane/AcOEt (3:7) to (0: 10) followed by AcOEt/methanol (10:0) to (9:1) and afforded the desired product (32 mg, 24%) as a brown solid.

¹ H-NMR (250 MHz, CDCl₃): 2.28-2.41 (m, 4H, 4-CH), 3.11-3.19 (m, 2H, 2-CH), 3.23-3.38 (m, 2H, 2-CH), 3.39-3.48 (m, 2H, 2-CH), 3.56-3.68 (m, 1H, CH), 3.72-3.86 (m, 2H, 2-CH), 6.72 (dd, 1H, J=7.5 Hz, Harom), 6.90 (dd, 1H, J=7.5 Hz, Harom), 7.21-7.36 (m, 5H, 5Harom), 7.48 (dd, 2H, J=7.5 Hz, Harom), 7.73-7.84 (m, 3H, 3Harom), 8.24 (d, 1H, J1=5 Hz, Harom), HPLC 96%, Rt=2.13 min. MS (AP) m/z 416.30 (M+H).

### EXAMPLE 6

### 1-(4-methoxyphenyl)-3-[1-(2-phenylethyl)piperidin4-yl]imidazo[1,5-a]pyridine

To a solution of 1-(4-methoxyphenyl)-3-piperidin-4-ylimidazo[1,5-a]pyridine (synthesized according to General procedure A; step 1-4) (170 mg, 0.55 mmol) in dry acetonitrile (2 ml) was added Hunig's base (0.261 ml, 1.5 mmol) and 2-(bromoethyl)benzene (92.5 mg, 0.5 mmol). The reaction mixture was heated to reflux for two days. DCM (50 ml) was added and the solution washed with water (50 ml). The organic layers were combined, washed with brine, dried over MgSO₄ and concentrated. The residue was purified by flash chromatography on silica gel eluting with a mixture of methanol/AcOEt (1:9) and afforded the desired product (12.6 mg, 6%) as brown solid.

HPLC 99%, Rt=1.92 min. MS (AP) m/z 412.28 (M+H).

### GENERAL PROCEDURE B FOR EXAMPLE 7-40 (LIBRARY COMPOUNDS)

### DETAILS OF SYNTHESIS OF EXAMPLES 7-40

As mentioned above, the process for the preparation of the compounds is as follows: 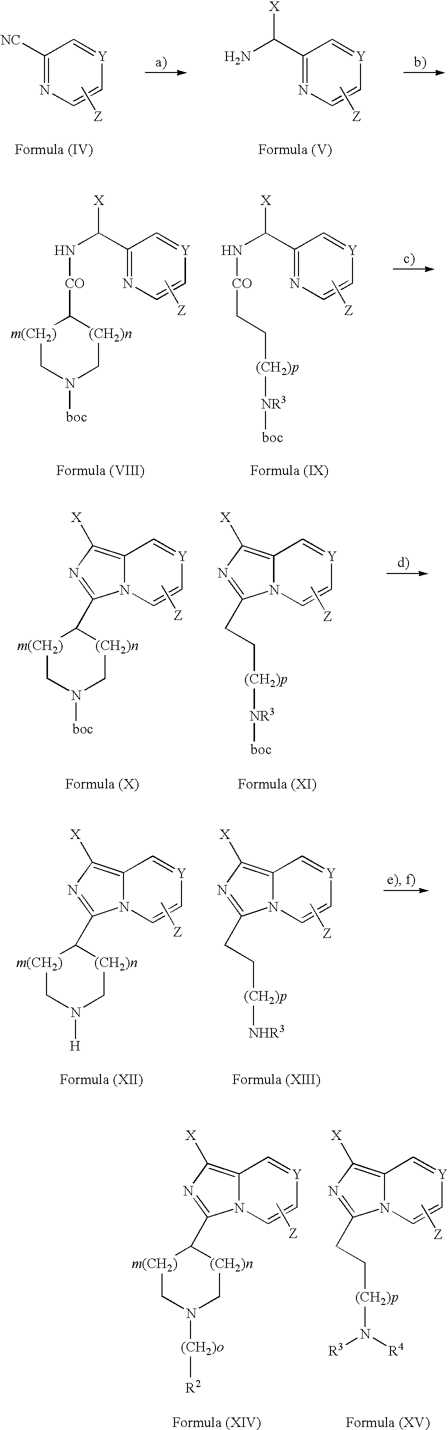
Step A)—Synthesis Of Pyridinylmethylamines Of Formula (V)

The cyanopyridine of Formula (IV) (0.1 mol) was dissolved in dry toluene (300 ml) and cooled to 0-5° C. The Grignard reagent (0.11 mol) was added dropwise over 30 minutes to give a thick creamy precipitate. The reaction was stirred for a further 30 minutes at 0-5° C. then isobutanol (120 ml) was added dropwise keeping the temperature below 0-5° C. to give a clear brown solution. The reaction was cooled to 0-5° C. and sodium borohydride (0.14 mol) added portionwise and the whole stirred at room temperature overnight. The reaction was quenched with methanol/water and concentrated in vacuo to remove the toluene. The mixture was extracted with DCM and the organics dried over magnesium sulfate before concentrating in vacuo. Purification was carried out by flash column chromatography on silica eluted with ethyl acetate and ethyl acetate/3% TEA mixtures. An alternative purification involved dissolving the residue in diethyl ether and extraction into dilute HCl. The acidic solution was washed three times with diethyl ether and then basified with 1N sodium hydroxide and the product extracted with diethyl ether. The organics were dried over magnesium sulfate and concentrated in vacuo to yield the pyridinylmethylamine of Formula (V).

Step B)—Synthesis Of Amides Of Formula (VIII) And (IX)

The BOC protected amino acid of Formula (VI) or (VII) (15 mmol) was dissolved in dry DCM (25 ml) and CDI (15 mmol) added. The reaction was stirred for 30 minutes and then a solution of the pyridinylmethylamine of Formula (V) (15 mmol) in DCM (5 ml) was added. The mixture was stirred overnight. The solution was washed with water, dried over magnesium sulfate and concentrated in vacuo to yield the desired amide of Formula (VII) or (IX). The amide was used without further purification.

Step c)—Cyclization to Give the Imidazopyridine of Formula (X) or (XI)

For Cyclic Amino Acids POCl ₃ (8.5 mmol) was added dropwise to a cooled (ice/water) solution of the amide of Formula (VIII) (7.2 mmol) and pyridine (44.5 mmol) in dry DCM (35 ml). The mixture was stirred overnight at room temperature. The mixture was washed with water (2×10 ml). The organics were dried over magnesium sulfate and concentrated in vacuo to yield the desired cyclised product of Formula (X). Purification was carried out by column chromatography on silica eluted with petrol:ethyl acetate

For Open Chain Amino Acids

The amide of Formula (IX) (2 mmol) was dissolved in dry DCM (10 ml) and pyridine (4 mmol) added. TFAA (2 mmol) was dissolved in dry DCM (2.5 ml) and added dropwise to the mixture at room temperature. The reaction was stirred for 1 h at room temperature. The mixture was washed with water (2×10 ml). The organics were dried over magnesium sulfate and concentrated in vacuo to yield the desired cyclised product of Formula (XI). Purification was carried out by column chromatography on silica eluted with petrol:ethyl acetate

Step d)—Deprotection to Give a Compound of Formula (XII) and (XIM

The BOC protected amine of Formula (X) or (XI) (8.86 mmol) was dissolved in (4N) methanolic HCl (15 ml) and stirred overnight at room temperature. The solvent was removed in vacuo and the solid residue triturated with diethyl ether. The solid was removed by filtration and dried to give the amine hydrochloride. The compounds were stored as the HCl salt and then converted to the free base of Formula (XII or (XIII) by aqueous sodium hydroxide for further reaction.

Step e)—Alkylation to Give an Amine of Formula (XIV) or (XV) Via Displacement of a Leaving Group

The free amine of Formula (XII) or (XIII) (0.2 mmol), alkylating agent (e g a bromide or methanesulfonate) (0.2 mmol) and Hunig's base (0.2 mmol) were heated in MIBK (2 ml) at 100° C. for 5 hours. The reaction was cooled and water added. The mixture was extracted with ethyl acetate (2×1 ml). The organics were dried over magnesium sulfate and concentrated in vacuo to yield the desired product of Formula (XIV) or (XV). Purification was carried out by automated preparative HPLC.

The reaction mixture was dissolved in DMSO (˜1.5 ml). This solution was loaded onto a 10 mm Xterra MS C₁₈ column at room temperature and eluted with the following gradient

Eluant A 0.05% DEA in water
Eluant B 0.05% DEA in ACN

| min | A | B |
|---|---|---|
| 0 | 95% | 5% |
| 1 | 95% | 5% |
| 6 | 5% | 95% |
| 10 | 5% | 95% |

re-equilibrate to 95% A prior to next injection

Sample collection was triggered by U.V. absorbance, (thresholds set appropriated for the specific plates). The collected samples were analysed by LC-MS to ascertain the identity and purity of the constituents.

Step f)—Alkylation to Give an Amine of Formula (XIV) or (XV) Via Reductive Amination

The free amine of Formula (XII) or (XIII), aldehyde/ketone and sodium triacetoxyborohydride were mixed and shaken overnight at room temperature. The reaction was diluted with DCM, washed with IM sodium bicarbonate solution and then water. The aqueous phase was back extracted with DCM. The organics were combined and concentrated. Purification was carried out by automated preparative HPLC, to give a product of Formula (XIV) or (XIV).

### EXAMPLE 7

### 1-(4-chlorophenyl)-7-methyl-3-piperidin4-ylimidazo[1,5-a]pyridine

Example 7 was synthesized according to general procedure B.

HPLC 96%, Rt=4.47 min. MS (AP) m/z 326 (M+H).

### EXAMPLE 8

### 1-(3-methylphenyl)-3-[1-(2-phenylethyl)piperidin4-yl]imidazo[1,5-a]pyridine

Example 8 was synthesized according to general procedure B.

HPLC 88%, Rt=5.60 min. MS (AP) m/z 396 (M+H).

### EXAMPLE 9

### 1-(2-methoxyphenyl)-3-[1-(2-phenylethyl)piperidin4-yl]imidazo[1,5-alpyridine

Example 9 was synthesized according to general procedure B.

HPLC 92%, Rt=5.09 min. MS (AP) m/z 412 (M+H).

### EXAMPLE 10

### 3-{1-[2-(4-fluorophenyl)ethyl]piperidin4-yl}-1-(2-methoxyphenyl)imidazo[1,5a]pyridine

Example 10 was synthesized according to general procedure B.

HPLC 93%, Rt=5.15 min. MS (AP) m/z 430 (M+H).

### EXAMPLE 11

### 7-chloro-1-(3-methoxyphenyl)-3-{1-[2-(3-methylphenyl)ethyl]piperidin4-yl}imidazo[1,5-a]pyridine

Example 11 was synthesized according to general procedure B.

HPLC 97%, Rt=6.07 min. MS (AP) m/z 461 (M+H).

### EXAMPLE 12

### 1-(3-methoxyphenyl)-3-{1-[2-(2-thienyl)ethyl]piperidin4-yl}imidazo[1,5-a]pyridine

Example 12 was synthesized according to general procedure B.

HPLC 85%, Rt=5.34 min. MS (AP) m/z 418 (M+H).

### EXAMPLE 13

### 7-chloro-3-[1-(2,3-dihydro-1 H-inden-2-yl)pyrrolidin-3-yl]-1-(4-methoxyphenyl)imidazo[1,5-a]pyridine

Example 13 was synthesized according to general procedure B.

HPLC 88%, Rt=5.55 min. MS (AP) m/z 444 (M+H).

### EXAMPLE 14

### 3-{1-[2-(2,6dimethoxyphenoxy)ethyl]piperidin4-yl}-1-(4-fluorophenyl)imidazo[1,5-a]pyridine

Example 14 was synthesized according to general procedure B.

HPLC 90%, Rt=5.09 min. MS (AP) m/z 476 (M+H).

### EXAMPLE 15

### 7-chloro-1-(3-methoxyphenyl)-3-[1-(2-phenylethyl)piperidin4-yl]imidazo[1, 5-a]pyridine

Example 15 was synthesized according to general procedure B.

HPLC 95%, Rt=5.78 min. MS (AP) m/z 446 (M+H).

### EXAMPLE 16

### 3-[1-(4-chlorophenyl)imidazo[1,5-a]pyridin-3-yl]-N-methylpropan-1-amine

Example 16 was synthesized according to general procedure B.

HPLC 96%, Rt=4.12 min. MS (AP) m/z 300 (M+H).

### EXAMPLE 17

### 3-(1-allylpiperidin-4-yl)-7-chloro-1-phenylimidazo[1,5-a]pyridine

Example 17 was synthesized according to general procedure B.

HPLC 97%, Rt=5.24 min. MS (AP) m/z 352 (M+H).

### EXAMPLE 18

### 3-{1-[3-(4-fluorophenoxy)propyl]piperidin-3-yl}-1-(2-methoxyphenyl)imidazo[1,5-a]pyridine

Example 18 was synthesized according to general procedure B.

HPLC 92%, Rt=4.61 min. MS (AP) m/z 460 (M+H).

### EXAMPLE 19

### 1-(4-fluorophenyl)-3-[1-(2-phenylethyl)piperidin4-yl]imidazo[1,5-a]pyridine

Example 19 was synthesized according to general procedure B.

HPLC 88%, Rt=5.39 min. MS (AP) m/z 400 (M+H).

### EXAMPLE 20

### 1-(1,3-benzodioxol-5-yl)-7-chloro-3-{1-[2-(3,4,5-trimethoxyphenyl)ethyl]piperidin4-yl}imidazo[1,5-a]pyridine

Example 20 was synthesized according to general procedure B.

HPLC 96%, Rt=5.32 min. MS (AP) m/z 551 (M+H).

### EXAMPLE 21

### 7-chloro-3-{1-[2-(1H-indol-3-yl)ethyl]piperidin-4-yl}-1-(3-methoxyphenyl)imidazo[1,5-alpyridine

Example 21 was synthesized according to general procedure B.

HPLC 94%, Rt=5.51 min. MS (AP) m/z 486 (M+H).

### EXAMPLE 22

### 1-(3-methoxyphenyl)-3-[1-(3-phenylpropyl)pyrrolidin-3-yl]imidazo[1,5-a]pyridine

Example 22 was synthesized according to general procedure B.

HPLC 86%, Rt=5.78 min. MS (AP) m/z 412 (M+H).

### EXAMPLE 23

### 3-{1-[2-(5-methoxy-1H-indol-3-yl)ethyl]piperidin4-yl}-1-(2-methoxyphenyl)imidazo[1,5-a]pyridine

Example 23 was synthesized according to general procedure B.

HPLC 88%, Rt=4.72 min. MS (AP) m/z 481 (M+H).

### EXAMPLE 24

### 2,3-dihydro-1H-inden-2-yl(methyl){3-[1-(3-methylphenyl)imidazo[1,5-a]pyridin-3-yl]propyl}amine

Example 24 was synthesized according to general procedure B.

HPLC 93%, Rt=5.04 min. MS (AP) m/z 396 (M+H).

### EXAMPLE 25

### 2,3-dihydro-1H-inden-2-yl{3-[1-(3-methoxyphenyl)imidazo[1,5-a]pyridin-3-yl]propyl}methylamine

Example 25 was synthesized according to general procedure B.

HPLC 95%, Rt=4.57 min. MS (AP) m/z 412 (M+H).

### EXAMPLE 26

### 7-chloro-3-{1-[2-(4-fluorophenyl)ethyl]piperidin-4-yl}-1-(2-methoxyphenyl)imidazo[1,5-a]pyridine

Example 26 was synthesized according to general procedure B.

HPLC 98%, Rt=5.67 min. MS (AP) m/z 464 (M+H).

### EXAMPLE 27

### 2,3-dihydro-1H-inden-2-yl{2-[1-(4-fluorophenyl)imidazo[1,5-a]pyrazin-3-yl]ethyl}methylamine

Example 27 was synthesized according to general procedure B.

HPLC 87%, Rt=3.68 min. MS (AP) m/z 387 (M+H).

### EXAMPLE 28

### 3-(1,3-benzodioxol-5-yl)-N-{2-[1-(2-methoxyphenyl)imidazo[1,5-a]pyridin-3-yl]ethyl}-2-methylpropan-1-amine (racemic)

Example 28 was synthesized according to general procedure B.

HPLC 89%, Rt=5.15 min. MS (AP) m/z 444 (M+H).

### EXAMPLE 29

### 2,3-dihydro-1H-inden-2-yl(methyl)[3-(1-phenylimidazo[1,5-a]pyridin-3-yl)propyl]amine

Example 29 was synthesized according to general procedure B.

HPLC 90%, Rt=4.65 min. MS (AP) m/z 382 (M+H).

### EXAMPLE 30

### 1-(3-methylphenyl)-3-[1-(3-phenoxypropyl)piperidin-3-yl]imidazo[1,5-a]pyridine

Example 30 was synthesized according to general procedure B.

HPLC 98%, Rt=5.53 min. MS (AP) m/z 426 (M+H).

### EXAMPLE 31

### 3-{1-[2-(4-bromophenyl)ethyl]piperidin4-yl}-7-methyl-1-phenylimidazo[1,5-a]pyridine

Example 31 was synthesized according to general procedure B.

HPLC 80%, Rt=6.05 min. MS (AP) m/z 474 (M+H).

### EXAMPLE 32

### 1-(4-fluorophenyl)-3-[1-(1-phenylethyl)piperidin-4-yl]imidazo [1,5-a]pyridine (racemic) Example 32 was synthesized according to general procedure B.

HPLC 96%, Rt=5.58 min. MS (AP) m/z 400 (M+H).

### EXAMPLE 33

### 3-(1-allylpiperidin-4-yl)-1-(4-chlorophenyl)imidazo[1,5-a]pyridine

Example 33 was synthesized according to general procedure B.

HPLC 97%, Rt=5.21 min. MS (AP) m/z 352 (M+H).

### EXAMPLE 34

### 2-{2-[4-(1-biphenyl-4-ylimidazo[1,5-a]pyridin-3-yl)piperidin-1-yl]ethyl}hexahydro-1H-isoindole-1,3(2H)-dione

Example 34 was synthesized according to general procedure B.

HPLC 91%, Rt=5.84 min. MS (AP) m/z 533 (M+H).

### EXAMPLE 35

### 3-[1-(2,3-dihydro-1H-inden-2-yl)pyrrolidin-3-yl]-1-(3-methoxyphenyl)imidazol1,5-a]pyridine

Example 35 was synthesized according to general procedure B.

HPLC 88%, Rt=5.65 min. MS (AP) m/z 410 (M+H).

### EXAMPLE 36

### 3-[1-(3-phenylpropyl)piperidin-4-yl-1-(2-thienyl)imidazo[1,5-a]pyridine

Example 36 was synthesized according to general procedure B.

HPLC 96%, Rt=5.36 min. MS (AP) m/z 402 (M+H).

### EXAMPLE 37

### 7-chloro-1-(2-methoxyphenyl)-3-{1-[2-(3,4,5-trimethoxyphenyl)ethyllpiperidin-4-yl}imidazo[1,5-a]pyridine

Example 37 was synthesized according to general procedure B.

HPLC 96%, Rt=5.28 min. MS (AP) m/z 537 (M+H).

### EXAMPLE 38

### 1-(1,3-benzodioxol-5-yl)-7-chloro-3-{1-[2-(5-methoxy-1H-indol-3-yl)ethyl]piperidin-4-yl}imidazo[1,5-a]pyridine

Example 38 was synthesized according to general procedure B.

HPLC 96%, Rt=5.25 min. MS (AP) m/z 530 (M+H).

### EXAMPLE 39

### 1-(1,3-benzodioxol-5-yl)-7-chloro-3-{1-[2-(2,6dimethoxyphenoxy)ethyl]piperidin4-yl}imidazo[1,5-a]pyridine

Example 39 was synthesized according to general procedure B.

HPLC 98%, Rt=5.41 min. MS (AP) m/z 537 (M+H).

### EXAMPLE 40

### 1-(1,3-benzodioxol-5-yl)-3-{1-[2-(5-methoxy-1H-indol-3-yl)ethyl]piperidin4-yl}imidazo[1,5-a]pyridine

Example 40 was synthesized according to general procedure B.

HPLC 87%, Rt=4.83 min. MS (AP) m/z 495 (M+H).

### EXAMPLE 41

Preparation of Tablets

| | Ingredients | mg/tablet |
|---|---|---|
| 1. | Active compound of formula (I) | 10.0 |
| 2. | Cellulose, microcrystalline | 57.0 |
| 3. | Calcium hydrogen phosphate | 15.0 |
| 4. | Sodium starch glycolate | 5.0 |
| 5. | Silicon dioxide, colloidal | 0.25 |
| 6. | Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

Primary screening and IC₅₀ determination

CHO cells expressing 5-HT_{2A} receptors seeded in 384 well plates are pre-loaded with Fluo-4AM fluorescent dye and then incubated with compound (10 μM for primary screen) for 15 min. Fluorescent intensity is recorded using a Fluorometric imaging plate reader (FLIPR384, Molecular Devices) and inhibition of the peak response evoked by 5-HT (EC₇₀ concentration) is calculated.

IC₅₀ determinations are performed utilizing the same finctional assay as described for primary screening (15 min antagonist compound pre-incubation), applying the compounds in the dose range of 3 nM to 10 μM.

In Vitro Receptor Pharmacology—Selectivity Determinations

The affinity constants of compounds were determined using recombinant human serotonin receptors stably expressed in fibroblast cell lines (CHO or HEK293), measuring the ability of the compounds to displace radio-labelled tracers using scintillation proximity assays or filter binding assays. For 5-HT_{1B} , 5-HT_{2B} and 5-HT_{2C} receptor binding studies ³H-LSD was used as radio ligand, for 5-HT_{2 A and} 5-HT₆ ³H-5-HT was used as tracer, while the binding constant to 5-HT_{1A} was determined using ³H-8-OH-DPAT. The non-selective serotonin receptor antagonist mianserine was used as reference substance.

The activity at 5-HT_{2C} receptors was studied in a FLIPR based assay, measuring the effect of compounds on 10 nM 5-HT induced Ca²⁺-currents.

Biology Summary

The calculation of the Kᵢ values for the inhibitors was performed by use of Activity Base. The Ki value is calculated from IC₅₀ using the Cheng Prushoff equation (with reversible inhibition that follows the Michaelis-Menten equation): Kᵢ =IC₅₀ (1+[S]/Kₘ ) [Cheng, Y. C.; Prushoff, W. H. Biochem. Pharmacol. 1973, 22, 3099-3108]. The compounds of formula (1) exhibit IC₅₀ values for the 5-HT2A receptor in the range from 1 nM to 10 μM.

Seven 5-HT_{2A} antagonist lead compounds were identified in FLIPR-based functional screening of the 5-HT_{2A} receptor. Four of these compounds were tested in equilibrium displacement binding measurements. The results of this study show that Example 2 and Example 4 are high affinity ligands for the 5-HT_{2A} receptor subtype, with Kᵢ values of 6 and 14, respectively (n=3). Both these compounds appear to be at least 20 fold selective over five other serotonin receptors assayed (5-HT_{2C}, 5-HT_{2B}, 5-HT_{1A}, 5-HT₆ and 5-HT_{1B}). Furthermore, Example 2 and Example 4 appear highly selective at 5-HT_{2A} versus the 5-HT_{2C} receptor in terms of efficacy. Reversibility of inhibition of the 5-HT_{2A} response was demonstrated for all compounds tested.

| | Functional | | | | | | |
|---|---|---|---|---|---|---|---|
| | Kᵢ (nM) | Binding Kᵢ (nM) | | | | | |
| Example | 5-HT_{2A} | 5-HT_{1A} | 5-HT_{1B} | 5-HT_{2A} | 5-HT_{2B} | 5-HT_{2C} | 5-HT₆ |
| Example 2 | 20.6 | >1000 | >1000 | 5.9 | 444 | >1000 | >1000 |
| Example 4 | 8.2 | >1000 | >1000 | 13.6 | 267 | >1000 | >1000 |

The table shows the selectivity of two example compounds for the 5-HT2A over other serotonin-binding receptors.

A number of embodiments of the invention have been described. Nevertheless, it will be understood that various modifications may be made without departing from the spirit and scope of the invention. Accordingly, other embodiments are within the scope of the following claims.

1. A compound of the Formula (I) 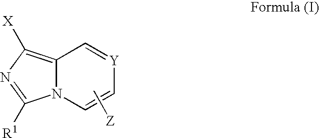
wherein
X is selected from aryl and heteroaryl, optionally independently substituted with one or more of C₁₋₆-alkyl, C₁₋₆-alkoxy, methylenedioxy, aryl, halogen, and halo-C₁₋₆-alkyl;
Y is selected from C-Z and N;
Z is selected from hydrogen, C₁₋₆-alkyl, C₁₋₆-alkoxy, and halogen;
R¹is either a group 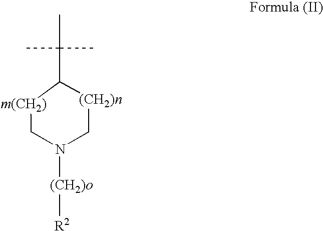
wherein
R² is selected from hydrogen; C₂₋₆-alkenyl, provided that o is 1; aryl optionally independently substituted with one or more of C₁₋₆-alkyl, C₁₋₆-alkoxy, halogen, cyano, and methylenedioxy, provided that o is 1-3; aryl-C₁₋₆-alkyl provided that o is 0; aryloxy optionally independently substituted with one or more of C₁₋₆-alkoxy and halogen, provided that o is 2-3; heteroaryl optionally independently substituted with one or more of C₁₋₆-alkyl and C₁₋₆-alkoxy; and heterocyclyl optionally independently substituted with one or more of C₁₋₆-alkyl and C₁₋₆-alkoxy;
m is 0 or 1;
n is 1 or 2;
o is 0,1,2, or 3; or
R¹is a group 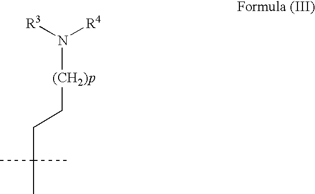
wherein
R³ is hydrogen or C₁₋₆-alkyl;
R⁴ is C₁₋₆-alkyl, aryl optionally independently substituted with one or more of C₁₋₆-alkyl and C₁₋₆-alkoxy; or heteroaryl-C₁₋₆-alkyl;
p is 0 or 1; and
pharmaceutically acceptable salts, hydrates, solvates, geometrical isomers, tautomers, optical isomers, and prodrug forms thereof.

2. A compound according to claim 1, wherein X is selected from: phenyl, optionally independently substituted with one or more of methyl, methoxy, methylenedioxy, phenyl, chloro, fluoro, and trifluoromethyl; and thienyl.

3. A compound according to claim 1, wherein X is selected from phenyl, 3-methylphenyl, 2-methoxyphenyl, 3-methoxyphenyl, 4-methoxyphenyl, 3,4-methylenedioxyphenyl, 1,1-biphenyl-4-yl, 4-chlorophenyl, 4-fluorophenyl, 2-thienyl, and 4-trifluoromethylphenyl.

4. A compound according to claim 1, wherein Z is selected from hydrogen, methyl, chloro, and methoxy.

5. A compound according to claim 1, wherein R² is selected from: hydrogen; vinyl; indanyl; phenyl, optionally independently substituted with one or more of methyl, methoxy, bromo, fluoro, cyano, and methylenedioxy; phenylethyl; phenoxy, optionally independently substituted with one or more of methoxy, fluoro, chloro, and bromo; indolyl, optionally independently substituted with one or more of methoxy; thienyl; and hexahydro-1H-isoindole-1,3(2H)-dione.

6. A compound according to claim 1, wherein R² is selected from hydrogen, vinyl, phenyl, 2-indanyl, 3-methylphenyl, 3,4,5-trimethoxyphenyl, 4-bromophenyl, 4-fluorophenyl, 1-phenylethyl, phenoxy, 2,6-dimethoxyphenoxy, 4-fluorophenoxy, 3-indolyl, 5-methoxy-3-indolyl, 2-thienyl, and hexahydro-1H-isoindole-1,3(2H)-dione.

7. A compound according to claim 1, wherein m+n is 1 or 2.

8. A compound according to claim 1, wherein R³ is selected from: hydrogen and methyl.

9. A compound according to claim 1, wherein R⁴ is selected from: methyl, 2-indanyl, and 2-methyl-3-(3,4-methylenedioxyphenyl)-n-propyl.

10. A compound according to claim 1, which is selected from:
2-(3-{4-[1-(4-fluorophenyl)imidazo[1 ,5-a]pyridin-3-yl]piperidin-1-yl}propyl)hexahydro-1H-isoindole-1,3(2H)-dione,
1-phenyl-3-[1-(2-phenylethyl)piperidin-4-yl]imidazo[1,5-a]pyridine,
3-{1-[2-(4-fluorophenyl)ethyl]piperidin-4-yl}-1-(3-methoxyphenyl)imidazo[1,5-a]pyridine,
7-methyl-1-phenyl-3-[1-(2-phenylethyl)piperidin-4-yl]imidazo[1,5-a]pyridine,
1-(4-chlorophenyl)-3-[1-(2-phenylethyl)piperidin-4-yl]imidazo[1,5-a]pyridine,
1-(4-methoxyphenyl)-3-[1-(2-phenylethyl)piperidin-4-yl]imidazo[1,5-a]pyridine,
1-(4-chlorophenyl)-7-methyl-3-piperidin-4-ylimidazo[1,5-a]pyridine,
1-(3-methylphenyl)-3-[1-(2-phenylethyl)piperidin-4-yl]imidazo[1,5-a]pyridine,
1-(2-methoxyphenyl)-3-[1-(2-phenylethyl)piperidin-4-yl]imidazo[1,5-a]pyridine,
3-{1-[2-(4-fluorophenyl)ethyl]piperidin-4-yl}-1-(2-methoxyphenyl)imidazo[1,5-a]pyridine,
7-chloro-1-(3-methoxyphenyl)-3-{1-[2-(3-methylphenyl)ethyl]piperidin-4-yl}imidazo[1,5-a]pyridine,
1-(3-methoxyphenyl)-3-{1-[2-(2-thienyl)ethyl]piperidin-4-yl}imidazo[1,5-a]pyridine,
7-chloro-3-[1-(2,3-dihydro-1H-inden-2-yl)pyrrolidin-3-yl]-1-(4-methoxyphenyl)imidazo[1,5-a]pyridine,
3-{1-[2-(2,6-dimethoxyphenoxy)ethyl]piperidin-4-yl}-1-(4-fluorophenyl)imidazo[1 ,5-a]pyridine,
7-chloro-1-(3-methoxyphenyl)-3-[1-(2-phenylethyl)piperidin-4-yl]imidazo[1,5-a]pyridine,
3-[1-(4-chlorophenyl)imidazo[1,5-a]pyridin-3-yl]-N-methylpropan-1-amine,
3-(1-allylpiperidin-4-yl)-7-chloro-1-phenylimidazo[1,5-a]pyridine,
3-{1-[3-(4-fluorophenoxy)propyl]piperidin-3-yl}-1-(2-methoxyphenyl)imidazo[1 ,5-a]pyridine,
1-(4-fluorophenyl)-3-[1-(2-phenylethyl)piperidin-4-yl]imidazo[1,5-a]pyridine,
1-(1,3-benzodioxol-5-yl)-7-chloro-3- {1-[2-(3,4,5-trimethoxyphenyl)ethyl]piperidin-4-yl}imidazo[1,5-a]pyridine,
7-chloro-3-{1-[2-(1H-indol-3-yl)ethyl]piperidin-4-yl}-1-(3-methoxyphenyl)imidazo[1,5-a]pyridine,
1-(3-methoxyphenyl)-3-[1-(3-phenylpropyl)pyrrolidin-3-yl]imidazo[1,5-a]pyridine,
3-{1-[2-(5-methoxy-1H-indol-3-yl)ethyl]piperidin-4-yl}-1-(2-methoxyphenyl)imidazo[1,5-a]pyridine,
2,3-dihydro-1H-inden-2-yl(methyl) {3-[1-(3-methylphenyl)imidazo[1,5-a]pyridin-3-yl]propyl}amine,
2,3-dihydro-1H-inden-2-yl{3-[1-(3-methoxyphenyl)imidazo[1,5-a]pyridin-3-yl]propyl}methylamine,
7-chloro-3-{1-[2-(4-fluorophenyl)ethyl]piperidin-4-yl}-1-(2-methoxyphenyl)imidazo[1,5-a]pyridine,
2,3-dihydro-1H-inden-2-yl{2-[1-(4-fluorophenyl)imidazo[1,5-a]pyrazin-3-yl]ethyl}methylamine,
3-(1,3-benzodioxol-5-yl)-N-{2-[1-(2-methoxyphenyl)imidazo[1,5-a]pyridin-3-yl]ethyl}-2-methylpropan-1-amine,
2,3-dihydro-1H-inden-2-yl(methyl)[3-(1-phenylimidazo[1,5-a]pyridin-3-yl)propyl]amine,
1-(3-methylphenyl)-3-[1-(3-phenoxypropyl)piperidin-3-yl]imidazo[1,5-a]pyridine,
3-{1-[2-(4-bromophenyl)ethyl]piperidin-4-yl}-7-methyl-1-phenylimidazo[1,5-a]pyridine,
1-(4-fluorophenyl)-3-[1-(1-phenylethyl)piperidin-4-yl]imidazo[1,5-a]pyridine,
3-(1-allylpiperidin-4-yl)-1-(4-chlorophenyl)imidazo[1,5-a]pyridine,
2-{2-[4-(1-biphenyl-4-ylimidazo[1,5-a]pyridin-3-yl)piperidin-1-yl]ethyl}hexahydro-1H-isoindole-1,3(2H)-dione,
3-[1-(2,3-dihydro-1H-inden-2-yl)pyrrolidin-3-yl]-1-(3-methoxyphenyl)imidazo[1,5-a]pyridine,
3-[1-(3-phenylpropyl)piperidin-4-yl]-1-(2-thienyl)imidazo[1,5-a]pyridine,
7-chloro-1-(2-methoxyphenyl)-3-{1-[2-(3,4,5-trimethoxyphenyl)ethyl]piperidin-4-yl}imidazo[1,5-a]pyridine,
1-(1,3-benzodioxol-5-yl)-7-chloro-3- {1-[2-(5-methoxy-1 H-indol-3-yl)ethyl]piperidin-4-yl}imidazo[1,5-a]pyridine,
1-(1,3-benzodioxol-5-yl)-7-chloro-3- {1-[2-(2,6-dimethoxyphenoxy)ethyl]piperidin-4-yl}imidazo[1,5-a]pyridine, and
1-(1,3-benzodioxol-5-yl)-3-{1-[2-(5-methoxy-1H-indol-3-yl)ethyl]piperidin-4-yl}imidazo[1,5-a]pyridine.

11. A process for the preparation of a compound according to claim 1, which process comprises the following steps:
a) reaction of a compound of Formula (IV) 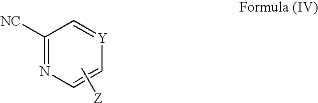
wherein
Y is selected from C—Z and N;
Z is selected from hydrogen, C₁₋₆-alkyl, C₁₋₆-alkoxy, and halogen;
with a Grignard reagent of Formula X-MgBr and then reduction with a reducing agent such as sodium borohydride
wherein
X is selected from aryl and heteroaryl, optionally independently substituted with one or more of C₁₋₆-alkyl, C₁₋₆-alkoxy, methylenedioxy, aryl, halogen, and halo-C₁₋₆-alkyl;
to give a compound of Formula (V) 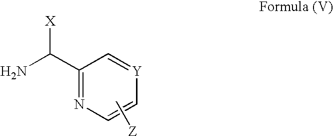
wherein
wherein X, Y, and Z are as defined above,
b) amidation by reaction of the compound of Formula (V) with either a carboxylic acid of Formula (VI) or of Formula (VII) in the presence of a coupling agent such as carbonyldiimidazole 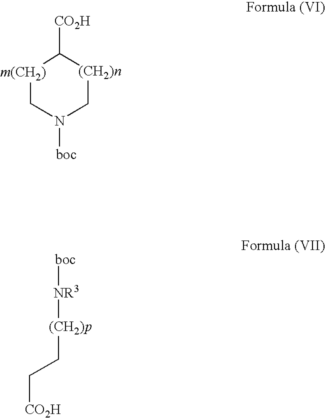
wherein
m is 0 or 1;
n is 1 or 2;
p is 0 or 1;
R³ is hydrogen or C₁₋₆-alkyl;
to give a compound of Formula (VIII) and (IX), respectively, 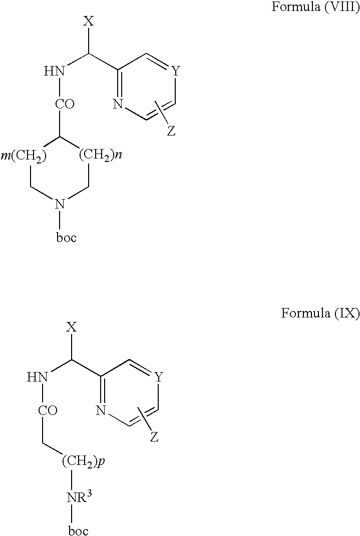
wherein X, Y, Z, m, n, p, and R³ are as defined above,
c) cyclization of the compound of Formula (VIII) with phosphorous oxychloride or the compound of Formula (IX) with trifluoroacetic anhydride, respectively, to give a compound of Formula (X) or (XI), respectively, 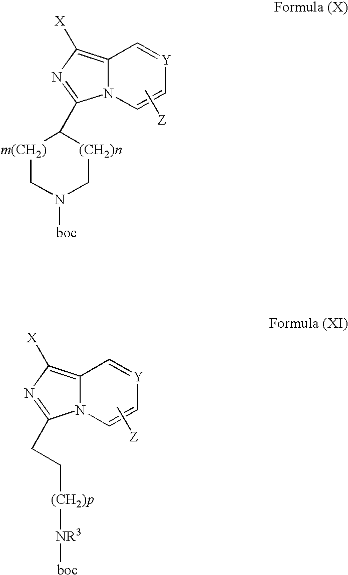
wherein X, Y, Z, m, n, p, and R³ are as defined above,
d) deprotection of the compound of Formula (X) or (XI), respectively, under acidic conditions, to give compounds of Formula (XII) or (XIII), respectively, 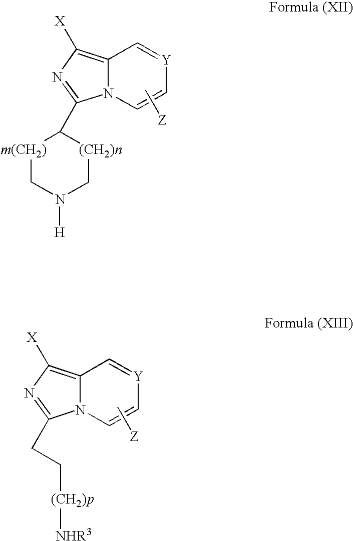
wherein X, Y, Z, m, n, p, and R³ are as defmed above; and, optionally, either of steps e) and f)
e) alkylation of the compound of Formula (XII) or (XIII), respectively, via displacement of a leaving group according to e1 ) and e2):
e1) reaction of the compound of Formula (XII) with an alkylating agent of the Formula R²—(CH₂)ₒ-LG in the presence of a tertiary amine such as N-ethyldiisopropylamine, wherein R² is selected from C₂₋₆-alkenyl, provided that o is 1; aryl optionally independently substituted with one or more of C₁₋₆-alkyl, C ₁₋₆-alkoxy, halogen, cyano, and methylenedioxy, provided that o is 1-3; aryl-C₁₋₆-alkyl, provided that o is 0; aryloxy optionally independently substituted with one or more of C₁₋₆-alkoxy and halogen, provided that o is 2-3; heteroaryl optionally independently substituted with one or more of C₁₋₆-alkyl and C₁₋₆-alkoxy; or heterocyclyl optionally independently substituted with one or more of C₁₋₆-alkyl and C₁₋₆-alkoxy, o is 0, 1, 2, or 3, and LG is a leaving group, to give a compound of Formula (XIV); or
e2) reaction of the compound of Formula (XIII) with an alkylating agent of the Formula R⁴-LG in the presence of N-ethyldiisopropylamine, wherein R⁴ is aryl optionally independently substituted with one or more of C₁₋₆-alkyl and C₁₋₆-alkoxy, or heteroaryl-C₁₋₆-alkyl; and LG is as defined above, to give a compound of Formula (XV) 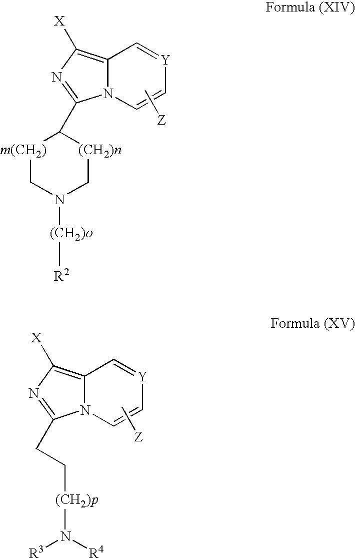
wherein X, Y, Z, m, n, o , p, R², R³, and R⁴ are as defined above;
f) alkylation of the compound of Formula (XII) or (XIII), respectively, via reductive amination according to f1) and f2):
f1) reaction of the compound of Formula (XII) with an aldehyde of the formula R²—(CH₂)_{q}—CHO, wherein R² is as defined above and q is 1-2, acetophenone or 2-indanone then a reducing agent such as sodium triacetoxyborohydride, to give a compound of Formula (XIV); or
f2) reaction of the compound of Formula (XIII) with an aldehyde of the formula R⁵—CHO, wherein R⁵ is heteroaryl-C₁₋₆-alkyl, preferably 1-methyl-2-(3,4-methylenedioxyphenyl)ethyl, or 2-indanone and then a reducing agent such as sodium triacetoxyborohydride, to give a compound of Formula (XV).

12. A pharmaceutical formulation comprising a compound according to claim 1 in combination with a pharmaceutically acceptable diluent or carrier.

13. A method for the prophylaxis or treatment of a 5-HT_{2A} receptor-related disorder, which comprises administering to a subject in need of such treatment an effective amount of a compound according claim 1.

14. The method according to claim 13, wherein the disorder is selected from schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence.

15. A method for modulating 5-HT_{2A} receptor activity, which comprises administering to a subject in need of such treatment an effective amount of a compound according to claim 1.
